(19) United States
(12) Patent Application Publication
(10) Pub. No.: US 2019/0274549 A1
(43) Pub. Date: Sep. 12, 2019

(54) PHOTOACOUSTIC IMAGING APPARATUS, METHOD FOR ACQUIRING INFORMATION, AND PROGRAM

(71) Applicant: CANON KABUSHIKI KAISHA, Tokyo (JP)

(72) Inventor: Yukio Furukawa, Sagamihara-shi (JP)

(73) Assignee: CANON KABUSHIKI KAISHA, Tokyo (JP)

(21) Appl. No.: 16/345,054

(22) PCT Filed: Oct. 19, 2017
(86) PCT No.: PCT/JP2017/037855

§ 371 Date: Apr. 25, 2019

(30) Foreign Application Priority Data
Oct. 26, 2016 (JP) 2016-209922

(51) Int. Cl. A61B 5/00

(52) U.S. Cl. CPC A61B 5/0095; A61B 5/7203

(57) ABSTRACT

A photoacoustic imaging apparatus includes a light irradiation unit, a receiving unit, and a processing unit. The light irradiation unit irradiates an object with first light for generating a photoacoustic wave corresponding to a positive coding element constituting a coding sequence and irradiates the object with second light for generating a photoacoustic wave corresponding to a negative coding element constituting the coding sequence. The receiving unit outputs a first signal by receiving the photoacoustic wave corresponding to the positive coding element, the photoacoustic wave being generated by irradiating the object with the first light, and outputs a second signal by receiving the photoacoustic wave corresponding to the negative coding element, the photoacoustic wave being generated by irradiating the object with the second light. The processing unit obtains a decoded signal by performing a decoding process on the first signal and the second signal based on information on the coding sequence.

### TECHNICAL FIELD

The present disclosure relates to a photoacoustic imaging apparatus using a photoacoustic effect.

### BACKGROUND ART

Research on optical imaging apparatuses that apply light onto an object to image information on the interior of the object acquired on the basis of the irradiation light is being actively pursued in the medical field. One of the optical imaging techniques is photoacoustic tomography (PAT). In the PAT, light emitted from a light source is applied to an object, and acoustic waves generated from tissue that has absorbed the energy of the light propagated and diffused in the object are detected. The phenomenon of acoustic wave generation is referred to as a photoacoustic effect, and the generated acoustic waves are referred to as photoacoustic waves. The acoustic waves are generally ultrasonic waves.

PTL 1 discloses irradiation of an object with a pulse train in which a plurality of pulsed lights are arranged. PTL 1 also discloses reconstruction of image data based on photoacoustic waves generated in an object by light irradiation.

### CITATION LIST

### Patent Literature

PTL 1: Japanese Patent Laid-Open No. 2014-39801

### SUMMARY OF INVENTION

### Technical Problem

In the case of photoacoustic tomography using a plurality of times of light irradiation, typically, after reception of photoacoustic waves generated by light irradiation is completed, the next light irradiation is performed, and reception of photoacoustic waves that are generated next is started. However, with this method, the number of times of light irradiation per unit time is limited. This makes it difficult to improve the signal-to-noise (S/N) ratios of the received signals of the photoacoustic waves obtained per unit time.

The present disclosure improves the S/N ratios of the received signals of photoacoustic waves obtained per unit time in a photoacoustic imaging apparatus using photoacoustic waves generated by a plurality of times of light irradiation.

A photoacoustic imaging apparatus includes a light irradiation unit, a receiving unit, and a processing unit. The light irradiation unit irradiates an object with first light for generating a photoacoustic wave corresponding to a positive coding element constituting a coding sequence and irradiates the object with second light for generating a photoacoustic wave corresponding to a negative coding element constituting the coding sequence. The receiving unit outputs a first signal by receiving the photoacoustic wave corresponding to the positive coding element, the photoacoustic wave being generated by irradiating the object with the first light, and outputs a second signal by receiving the photoacoustic wave corresponding to the negative coding element, the photoacoustic wave being generated by irradiating the object with the second light. The processing unit obtains a decoded signal by performing a decoding process on the first signal and the second signal based on information on the coding sequence.

Further features of the present invention will become apparent from the following description of exemplary embodiments with reference to the attached drawings.

### BRIEF DESCRIPTION OF DRAWINGS

FIG. 1A is a graph schematically illustrating light intensity corresponding to a positive coding element and the received signal of the photoacoustic wave.

FIG. 1B is a graph schematically illustrating light intensity corresponding to a negative coding element and the received signal of the photoacoustic wave.

FIG. 2A is a graph schematically illustrating light intensity corresponding to a coding sequence and the received signal of the photoacoustic wave.

FIG. 2B is a graph schematically illustrating light intensity corresponding to a coding sequence and the received signal of the photoacoustic wave.

FIG. 2C is a graph schematically illustrating light intensity corresponding to a coding sequence and the received signal of the photoacoustic wave.

FIG. 3 is a block diagram of a photoacoustic imaging apparatus according to an embodiment of the present disclosure.

FIG. 4 is a block diagram illustrating the configuration of a computer and its peripherals according to an embodiment of the present disclosure.

FIG. 5A is a graph illustrating a characteristic of a semiconductor laser.

FIG. 5B is a graph illustrating a characteristic of the semiconductor laser.

FIG. 5C is a graph illustrating a characteristic of the semiconductor laser.

FIG. 6A is a graph illustrating a received signal corresponding to a positive coding element.

FIG. 6B is a graph illustrating the reception characteristic of a transducer.

FIG. 6C is a graph illustrating a signal received by the transducer in FIG. 6B.

FIG. 7 is a graph illustrating a received signal corresponding to a positive coding element when rise time is changed.

FIG. 8 is a graph illustrating the relationship between rise time and a full width at half maximum (FWHM).

FIG. 9 is a graph illustrating a received signal corresponding to a positive coding element when fall time is changed.

FIG. 10 is a graph illustrating a received signal corresponding to a negative coding element when fall time is changed.

FIG. 11 is a graph illustrating the relationship between the time interval (period) of the reference timing and the correlation value of received signals corresponding to a positive coding element and a negative coding element.

FIG. 12A is a graph illustrating a driving current according to a first embodiment of the present disclosure.

FIG. 12B is a graph illustrating a received signal according to the first embodiment.

FIG. 12C is a graph illustrating a decoded signal according to the first embodiment.

FIG. 13A is a graph illustrating a received signal to which noise is added according to the first embodiment.

FIG. 13B is a graph illustrating a decoded signal of the received signal according to the first embodiment.

FIG. 14 is a diagram illustrating the configuration of a driving unit according to the first embodiment.

FIG. 15A is a graph illustrating a driving current according to a second embodiment of the present disclosure.

FIG. 15B is a graph illustrating a received signal obtained when the driving current in FIG. 15A is input.

FIG. 15C is a graph illustrating a driving current according to the second embodiment.

FIG. 15D is a graph illustrating a received signal obtained when the driving current in FIG. 15C is input.

FIG. 16A is a graph illustrating a decoded signal according to the second embodiment.

FIG. 16B is a graph illustrating a decoded signal according to the second embodiment.

FIG. 16C is a graph illustrating a decoded signal according to the second embodiment.

FIG. 17A is a graph illustrating a received signal to which noise is added according to the second embodiment.

FIG. 17B is a graph illustrating a received signal to which noise is added according to the second embodiment.

FIG. 18A is a graph illustrating a decoded signal of the received signal in FIG. 17A according to the second embodiment.

FIG. 18B is a graph illustrating a decoded signal of the received signal in FIG. 17B according to the second embodiment.

FIG. 18C is a graph illustrating a decoded signal in which the decoded signals in FIGS. 18A and 18B are added up.

### DESCRIPTION OF EMBODIMENTS

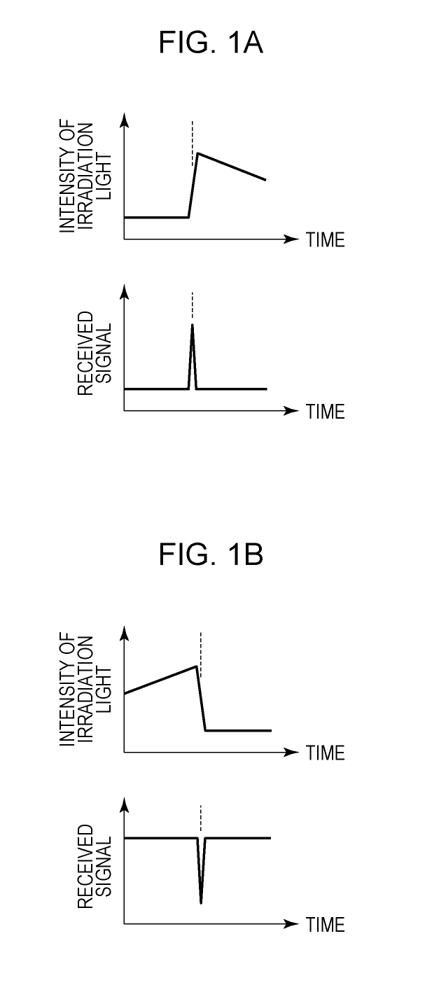

It is known in the art that, when light is applied to a substance, acoustic waves (also referred to as photoacoustic waves) are generated owing to a photoacoustic effect. A method of encoding based on a coding sequence including positive and negative coding elements by controlling irradiation light using a photoacoustic imaging apparatus that processes the received signals of the photoacoustic waves will be described. FIGS. 1A and 1B are graphs that schematically show temporal changes in the intensity of irradiation light and the level of the received signal of a photoacoustic wave generated by the irradiation light. Typically, as illustrated in FIG. 1A, when the temporal change in the intensity of the irradiation light is positive, a positive-level received signal can be obtained. In contrast, as illustrated in FIG. 1B, when the temporal change in the intensity of the irradiation light is negative, a negative-level received signal can be obtained. The level of the received signal increases as the change in the intensity of the irradiation light per unit time increases. In FIGS. 1A and 1B, the propagation time of photoacoustic waves from a sound source to a receiving unit is ignored.

The inventor has found out the present disclosure from the conception that the positive and negative of the level of the received signal can be controlled by controlling the positive and negative of a temporal change in the intensity of the irradiation light, as illustrated in FIGS. 1A and 1B. In other words, the inventor has found out that the positive and negative of coding elements constituting a coding sequence in encoding by controlling the positive and negative of a temporal change in the intensity of the irradiation light. For example, a coding sequence including positive and negative coding elements can be defined by combining light irradiation illustrated in FIG. 1A at which the coding element is {1} and light irradiation illustrated in FIG. 1B at which the coding elements is {−1}. In this specification, light for generating a photoacoustic wave corresponding to a positive coding element is referred to as “first light”. Light for generating a photoacoustic wave corresponding to a negative coding element is referred to as “second light”.

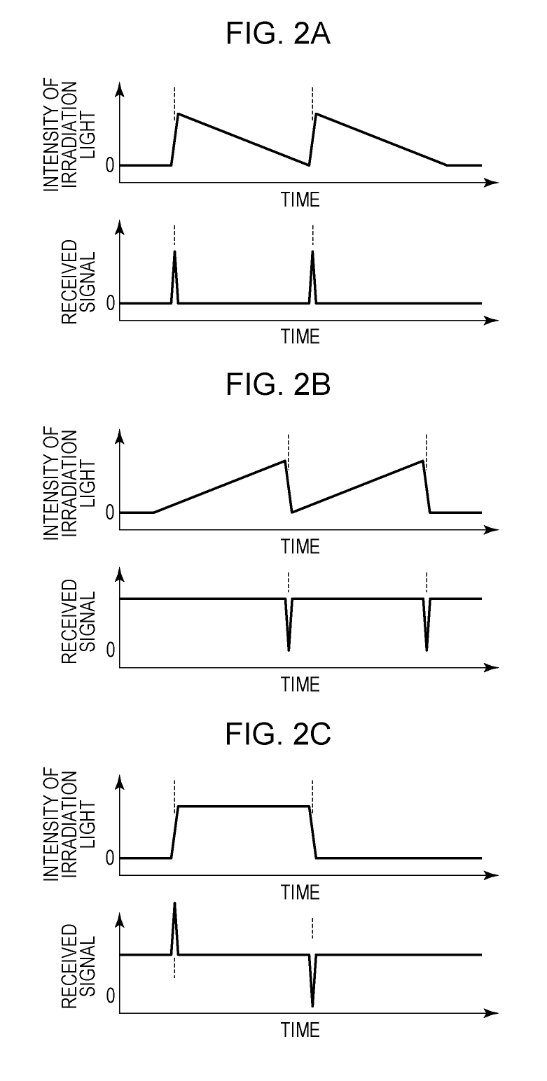

Examples of sequences of light irradiation corresponding to the coding sequences of several patterns will be described with reference to FIGS. 2A to 2C. The dotted lines in FIGS. 2A to 2C each indicate the reference timing of each coding element.

FIG. 2A is a graph that schematically illustrates temporal changes in the intensity of irradiation light and the level of the received signal of the photoacoustic wave corresponding to a coding sequence {1, 1}. The sequence of the irradiation light illustrated in FIG. 2A includes two consecutive lights (first lights) whose intensity sharply increases in a short time and gradually decreases with time. The timing at which the intensity increases sharply in a short time is adjusted to a reference timing corresponding to a positive coding element. For example, the timing at the center of the period during which the intensity sharply increases in a short time can be made to coincide with the reference timing. In this case a large positive received signal can be obtained at the reference timing. The large positive received signal is a signal corresponding to the positive coding element {1}.

FIG. 2B is a graph that schematically illustrates temporal changes in the intensity of irradiation light and the level of the received signal of the photoacoustic wave corresponding to a coding sequence {−1, −1}. The sequence of the irradiation light illustrated in FIG. 2B includes two consecutive lights (second lights) whose intensity gradually increases with time and sharply decreases in a short time. The timing at which the intensity sharply decreases in a short time is adjusted to a reference timing corresponding to a negative coding element. Specifically, the timing at the center of the period during which the intensity sharply decreases in a short time is made to coincide with the reference timing. In this case a large negative received signal can be obtained at the reference timing. The large negative received signal is a signal corresponding to the negative coding element {−1}.

FIG. 2C is a graph that schematically illustrates temporal changes in the intensity of irradiation light and the level of the received signal of the photoacoustic wave corresponding to a coding sequence {1, −1}. The sequence of the irradiation light illustrated in FIG. 2C is such that the first light illustrated in FIG. 2A is applied, and then the second light illustrated in FIG. 2B is applied. The irradiation timing is controlled so that the timing at which the intensity of the first light sharply increases corresponds to the reference timing of the positive coding element {1}, and the timing at which the intensity of the second light sharply decreases corresponds to the reference timing of the negative coding element {−1}. In this case, a large positive received signal and a large negative received signal can be obtained at the respective reference timings. Since the portion of the first light that gradually decreases with time and the portion of the second light that gradually increases with time overlap in time, the overlapping portion forms a rectangular wave as a result. Thus, when the positive and negative coding elements of the coding sequence are next to each other, an unnecessary photoacoustic wave is not generated by setting the light intensity between the reference timings substantially constant. This allows high-accuracy encoding using light irradiation. Although FIG. 2C illustrates an example of the coding sequence {1, −1}, the light intensity between the reference timings may be set substantially constant also for a coding sequence {−1, 1}. The temporal change in the light intensity between the reference timings may be assumed to be substantially constant as long as the temporal change is within a predetermined range in which a photoacoustic wave having a frequency out of the reception band of a transducer that receives the photoacoustic wave is generated.

In FIGS. 2A to 2C, the propagation time of photoacoustic waves from the sound source to the receiving unit is ignored.

Thus, decoding accuracy of decoding based on a coding sequence including positive and negative coding elements can be increased by performing light irradiation corresponding the coding sequence including positive and negative coding elements and encoding for the positive and negative coding elements. In particular, for semiconductor lasers and light-emitting diodes (LEDs) whose output light intensities are smaller than those of high-output light sources, such as a solid-state laser, the S/N ratio of the received signal is desirably improved by increasing the number of irradiation times per unit time. In such a case, by executing light irradiation for encoding on the basis of a coding sequence including positive and negative coding elements before reception of a photoacoustic wave generated first is completed, decoded signals with a high S/N ratio can be accurately obtained.

The acoustic waves generated owing to a photoacoustic effect according to an embodiment of the present disclosure is typically ultrasonic waves including sound waves and acoustic waves. The present disclosure can be applied to a photoacoustic imaging apparatus that obtains image data on the basis of photoacoustic waves generated by the photoacoustic effect.

A photographic image acquired by the photoacoustic imaging apparatus according to an embodiment of the present disclosure includes all images derived from photoacoustic waves generated by light irradiation. The photographic image is image data indicating the spatial distribution of at least one of object information such as the generated sound pressure (initial sound pressure), the optical absorption energy density, and the optical absorption coefficient of the photoacoustic waves, and the concentration (the degree of oxygen saturation) of a substance constituting the object.

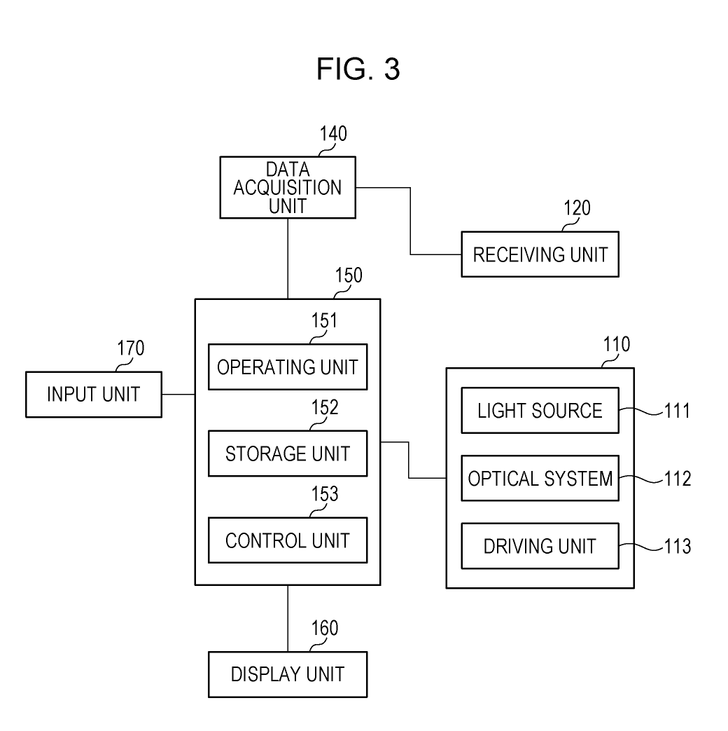

Referring to FIG. 3, the configuration of the photoacoustic imaging apparatus according to the present embodiment will be described hereinbelow. FIG. 3 is a schematic block diagram of the overall photoacoustic imaging apparatus. The photoacoustic imaging apparatus according to the present embodiment includes a light irradiation unit 110, a receiving unit 120, a data acquisition unit 140, a computer 150, a display unit 160, and an input unit 170.

The light irradiation unit 110 irradiates an object 100 with light, so that acoustic waves are generated from the object 100. The acoustic waves generated by a photoacoustic effect due to light are also referred to as photoacoustic waves. The receiving unit 120 receives the photoacoustic waves and outputs an electrical signal (a photoacoustic signal) which is an analog signal.

The data acquisition unit 140 converts the analog signal output from the receiving unit 120 to a digital signal and outputs the digital signal to the computer 150. The computer 150 stores the digital signal output from the data acquisition unit 140 as signal data derived from the photoacoustic wave.

The computer 150 serving as a processing unit performs processing (described later) on the stored digital signal to generate image data indicating a photographic image. The computer 150 also performs image processing on the acquired image data for display and thereafter outputs the image data to the display unit 160. The display unit 160 displays the photographic image. The user, such as a doctor or an operator, can make a diagnosis by checking the photographic image displayed on the display unit 160. The display image is stored in a memory in the computer 150 or a data management system connected to the modality over a network on the basis of a storage instruction from the user or the computer 150.

The computer 150 also controls driving of the components of the photoacoustic imaging apparatus. The display unit 160 may also display a graphic user interface (GUI) in addition to images generated by the computer 150. The input unit 170 is configured so that the user can input information. The user can perform operations such as instructions to start and terminate measurement and store a generated image.

The details of the components of the photoacoustic imaging apparatus according to the present embodiment will be described hereinbelow.

### Light Irradiation Unit 110

The light irradiation unit 110 includes a light source 111 that emits light, an optical system 112 that guides the light emitted from the light source 111 to the object 100, and a driving unit 113 that controls driving of the light source 111.

The light emitted from the light source 111 may have a pulse width of 1 ns or more and 100 ns or less. The light may have a wavelength within a range of about 400 nm to 1,600 nm. For imaging blood vessels at high resolution, light having a wavelength (400 nm or more and 700 nm or less) with large absorptance in blood vessels may be used. For imaging a deep part of a living organism, light having a wavelength (700 nm or more and 1,100 nm or less) with typically small absorptance in background tissue (water, fat, etc.) of a living organism may be used.

Examples of the light source 111 include a laser and a light-emitting diode. For measurement using lights with a plurality of wavelengths, a light source capable of emitting changing the wavelength of light may be used. In applying lights with a plurality of wavelengths to the object, a plurality of light sources that generate lights with different wavelengths may be provided, so that light can be alternately applied from the individual light sources. The plurality of light sources are also expressed as a single light source. Examples of the laser include a solid-state laser, a gas laser, a dye laser, a semiconductor laser, and other various lasers. For example, a pulse laser, such as a yttrium aluminum garnet (Nd:YAG) laser or an alexandrite laser, may be used as the light source. Alternatively, a titanium sapphire (Ti:sa) laser that uses a Nd:YAG laser light as excitation light or an optical parametric oscillator (OPO) laser may be used as the light source. Other examples of the light source 111 are a flash lamp and a light-emitting diode (LED). Still another example of the light source 111 is a microwave source.

For example, the light source 111 may be a semiconductor laser or an LED capable of emitting light following a saw tooth driving waveform (a driving current) with a frequency of 1 MHz or more. Alternatively, a wavelength-variable light source capable of emitting lights of different wavelengths may be used.

Examples of the optical system 112 include optical elements, such as a lens, a mirror, and an optical fiber. If the object 100 is a breast, the light output unit of the optical system 112 may be a diffuser panel for diffusing light to increase the beam diameter of the pulsed light. For a photoacoustic microscope, the light output unit of the optical system 112 may be a lens and focus the beam to increase the resolution. The light irradiation unit 110 may apply light directly from the light source 111 to the object 100 without the optical system 112.

The driving unit 113 generates a driving current (a current to be input to the light source 111) for driving the light source 111. The driving unit 113 may use a power source capable of temporally varying a current to be input to the light source 111. By controlling the output of the light source 111 with the driving unit 113, the light as illustrated in FIGS. 1A and 1B is generated to achieve encoding. The driving unit 113 may be controlled by a control unit 153 (described later) in the computer 150. The driving unit 113 may include a control unit for controlling the current value so that the control unit can control the input current. The relationship between the driving current and the intensity of the irradiation light will be described later.

### Receiving Unit 120

The receiving unit 120 includes a transducer that outputs an electrical signal by receiving acoustic waves and a supporter that supports the transducer.

Examples of a constituent member of the transducer include a piezoelectric ceramic material typified by piezoelectric zirconate titanate (PZT) (lead zirconate titanate) and a high-molecule piezoelectric membrane material typified by polyvinylidene fluoride (PVDF). Other elements other than the piezoelectric element may be used. For example, a capacitive micro-machined ultrasonic transducer (CMUT) or a transducer with a Fabry-Perot interferometer can be used. Any transducers capable of outputting an electrical signal by receiving acoustic waves can be employed. The signal obtained by the transducer is a time-resolved signal. In other words, the amplitude of the signal obtained by the transducer indicates a value based on a sound pressure received by the transducer at each time (for example a value proportional to the sound pressure).

A frequency component constituting the photoacoustic wave typically ranges from 100 KHz to 100 MHz, and a transducer capable of detecting these frequencies can be employed.

The supporter may be a supporter that supports a plurality of transducers on a flat surface or a curved surface called a 1D array, a 1.5D array, a 1.75D array, or a 2D array. An array in which a plurality of transducers are arranged in a curved surface is also referred to as a three-dimensional transducer array.

The receiving unit 120 may further include an amplifier that amplifies time-series analog signals output from the transducer. The receiving unit 120 may further include an analog-to-digital converter that converts time-series analog signals output from the transducer to time-series digital signals. In other words, the receiving unit 120 may include the data acquisition unit 140 (described later).

A plurality of transducers may be ideally disposed around the object 100 to detect acoustic waves at various angles. However, if the object 100 is so large that the transducers cannot be disposed so as to surround the entire periphery thereof, the transducers may be disposed on a hemispherical supporter to substantially surround the entire periphery. The arrangement and number of the transducers and the shape of the supporter may be optimized according to the object, and any type of receiving unit 120 can be employed in the present disclosure.

The space between the receiving unit 120 and the object 100 may be filled with a medium capable of propagating photoacoustic waves. An example of this medium is a material in which acoustic waves can propagate and the acoustic characteristics match at the interface with the object 100 and the transducers and which has the highest possible transmittance of the photoacoustic waves. For example, this medium can be water or ultrasonic wave gel.

In the case where the apparatus according to the present embodiment generates an ultrasonic image, in addition to the photographic image, by transmitting and receiving acoustic waves, the transducer may also function as a transmitting unit that transmits the acoustic waves. The transducer serving as a receiving unit and the transducer serving as a transmitting unit may be a single (common) transducer or separate transducers.

### Data Acquisition Unit 140

The data acquisition unit 140 includes an amplifier that amplifies an electrical signal, which is an analog signal output from the receiving unit 120, and an analog-to-digital converter that converts the analog signal output from the amplifier to a digital signal. The data acquisition unit 140 may be formed of a field programmable gate array (FPGA) chip. The digital signal output from the data acquisition unit 140 is stored in a storage unit 152 in the computer 150. The data acquisition unit 140 is also referred to as a data acquisition system (DAS). In this specification, the electrical signal includes an analog signal and a digital signal. The data acquisition unit 140 is connected to a light sensor mounted to the light output unit of the light irradiation unit 110. The data acquisition unit 140 may start processing in synchronism with emission of light from the light irradiation unit 110. Alternatively, the data acquisition unit 140 may start the processing in synchronism with an instruction given using a freeze button or the like.

### Computer 150

The computer 150 serving as an information processing unit includes an operating unit 151, the storage unit 152, and the control unit 153. The function of each component will be described in the description of the process flow.

A unit responsible for an operating function as the operating unit 151 may include a processor, such as a central processing unit (CPU) or a graphics processing unit (GPU), or an operating circuit, such as a field programmable gate array (FPGA) chip. The unit may include either a single processor or operating circuit or a plurality of processor or operating circuits. The operating unit 151 may process the received signal using various parameters, such as the sound velocity of the object and the sound velocity of a medium in which the acoustic wave propagates, sent from the input unit 170.

The storage unit 152 may be a non-transitory storage medium, such as a read only memory (ROM), a magnetic disk, or a flash memory. The storage unit 152 may be a volatile medium, such as a random access memory (RAM). A storage medium in which programs are stored is a non-transitory storage medium. The storage unit 152 may include either one storage medium or a plurality of storage media.

The storage unit 152 can store image data indicating a photographic image generated by the operating unit 151 using a method described later.

The control unit 153 includes an operating element, such as a CPU. The control unit 153 controls the operation of each component of the photoacoustic imaging apparatus. The control unit 153 may control each component of the photoacoustic imaging apparatus by receiving instruction signals to start measurement or the like according to various operations from the input unit 170. The control unit 153 reads program codes stored in the storage unit 152 and controls the operations of the components of the photoacoustic imaging apparatus.

The computer 150 may be a dedicated workstation. The components of the computer 150 may be different pieces of hardware. At least part of the components of the computer 150 may be a single piece of hardware.

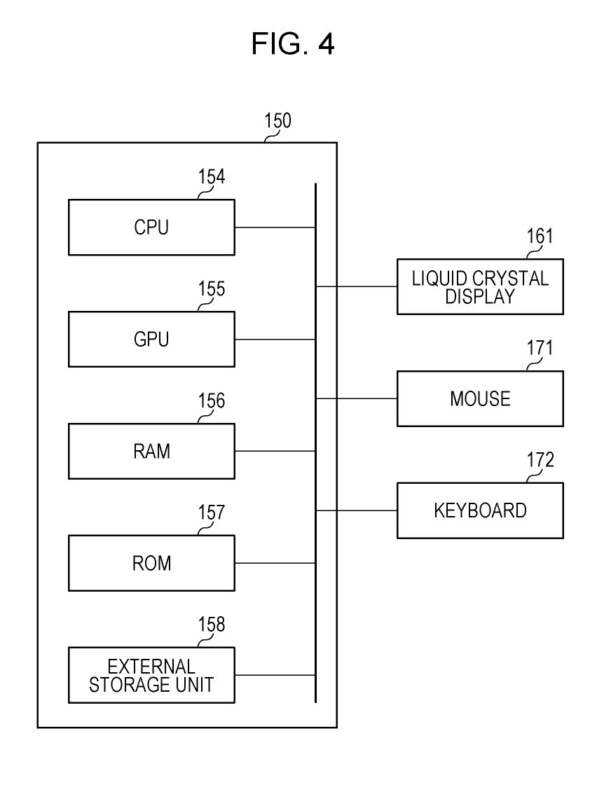

FIG. 4 illustrates a specific configuration example of the computer 150 according to the present embodiment. The computer 150 according to the present embodiment includes a CPU 154, a GPU 155, a RAM 156, a ROM 157, and an external storage unit 158. The computer 150 connects to a liquid crystal display 161 serving as the display unit 160 and a mouse 171 and a keyboard 172 serving as the input unit 170.

The computer 150 and the receiving unit 120 may be housed in a common casing. Part of signal processing may be performed by a computer housed in the casing, and the remaining signal processing may be performed by a computer disposed outside the casing. In this case, the computers disposed inside and outside the casing can be collectively referred to as a computer according to the present embodiment. In other words, the hardware constituting the computer may not be housed in one casing.

### Display Unit 160

Examples of the display unit 160 include a liquid crystal display and an organic electro luminescence (EL) display. The display unit 160 displays images and the value of a specified position based on object information obtained by the computer 150. The display unit 160 may display a GUI for operating images and the apparatus. The object information may be displayed after image processing (adjustment of the luminance value, etc.) is performed on the display unit 160 or the computer 150.

### Input Unit 170

The input unit 170 may be an operator operable console, such as a mouse and a keyboard. The display unit 160 may be a touch panel serving as the input unit 170.

The components of the photoacoustic imaging apparatus may be either separate units or a single unit. A least part of the components of the photoacoustic imaging apparatus may be a single unit.

### Object 100

The object 100, which is not a component of the photoacoustic imaging apparatus, will be described below. The photoacoustic imaging apparatus according to the present embodiment can be used to diagnose malignant tumors, vascular diseases, and so on of humans and animals and to follow up on chemotherapy. Therefore, conceivable examples of the object 100 include diagnostic target parts, such as the breasts, the organs, the vascular networks, the head, the neck, the abdomen, the extremities including fingers and toes of human bodies and animals. For example, for a human body, oxyhemoglobin, deoxyhemoglobin, blood vessels containing much oxyhemoglobin or deoxyhemoglobin, or new blood vessels formed in the vicinity of tumor may be the target light absorber. Furthermore, plaque on a carotid artery wall may be the target light absorber. Pigments, such as methylene blue (MB) and indocyanine green (ICG), gold fine particles, or externally introduced substances in which the above pigments or gold fine particles are accumulated or chemically modified may be the light absorber. A puncture needle or a light absorber put on a puncture needle may be the observation target.

### Received Signal of Photoacoustic Wave Corresponding to Coding Element {1}

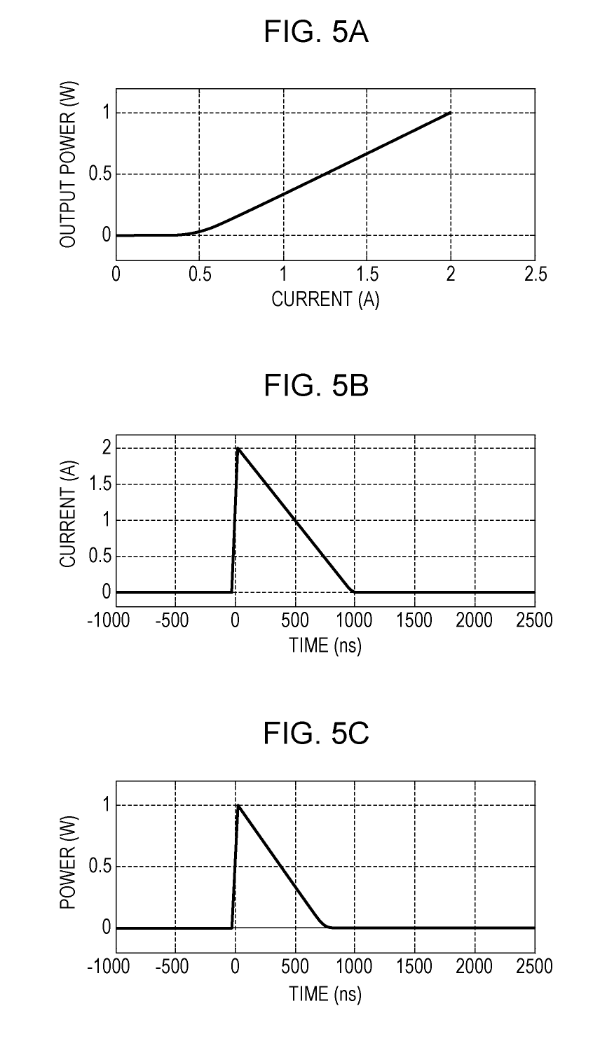

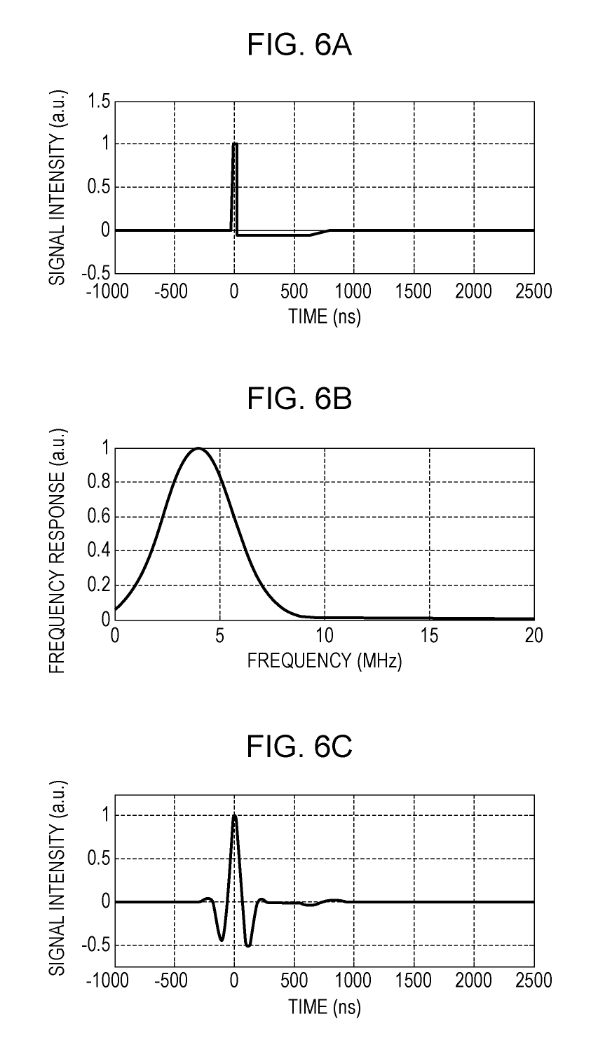

Irradiation light and the received signal of a photoacoustic wave corresponding to each coding element when the photoacoustic imaging apparatus according to the present embodiment is used will be considered. Referring to FIGS. 5A to 5C and FIGS. 6A to 6C, irradiation light and the received signal of a photoacoustic wave corresponding to a coding element {1} will be described. The data illustrated in FIGS. 5A to 6C and FIGS. 6A to 6C is data obtained by simulation.

FIG. 5A is a diagram illustrating the current-light output characteristic of a semiconductor laser serving as the light source 111. The threshold current of the semiconductor laser is 0.5 A, and the light output when a current of 2 A is input is 1 W. In the case of the semiconductor laser, the current-light output characteristic is typically substantially linear in a region where the current is equal to or greater than the threshold current. In other words, with the semiconductor laser, the temporal waveform of the input current is the temporal waveform of the light output (the intensity of the irradiation light).

FIG. 5B illustrates a driving current (a first driving current) for generating light corresponding to a positive coding element, in which the current value is increased from 0 A to 2 A in the time of 50 ns and is decreased from 2 A to 0 A in the time of 950 ns. In other words, as compared with the temporal change of the current at the timing corresponding to the positive coding element of the first driving current, the temporal change of the current at the other timing is smaller. Thus, the temporal change of the first light corresponding to the positive coding element at the reference timing corresponding to the positive coding element is larger than the temporal change of the light intensity at the other timing.

FIG. 5C illustrates light output when the semiconductor laser is driven by the driving current in FIG. 5B. This shows that the light is output substantially linearly with respect to the driving current, as described above.

FIG. 6A illustrates a received signal in the case where photoacoustic waves generated when a point-like light absorber is irradiated with light are received by a transducer with an infinite reception band. This is equal to the time derivative of the light output curve in FIG. 5C. Thus, a large positive received signal is obtained in accordance with a sharp rise in light output in a short time.

However, the reception band of the transducer is actually unlikely to be infinite and has some frequency characteristics. FIG. 6B illustrates the reception characteristics of a transducer having frequency characteristics of a central frequency of 4 MHz and frequencies of 2 to 6 MHz in a 6-dB band. FIG. 6C illustrates a received signal when photoacoustic waves generated by the light irradiation in FIG. 5C are received by a transducer having the reception characteristics illustrated in FIG. 6B. Thus, a large positive received signal can be obtained in correspondence with a sharp rise in light output in a short time even in consideration of the reception characteristic of the transducer. This large positive received signal is a received signal corresponding to a positive coding element (for example, the coding element {1}).

In FIGS. 6A to 6C, the time of propagation of the photoacoustic waves from the sound source to the receiving unit is ignored.

### Photoacoustic Wave Corresponding to Code Element {−1}

The received signal of a photoacoustic wave obtained by driving the semiconductor laser with a driving current (a second driving current) which is a current inverted from the driving current in FIG. 5B on the time axis has a waveform in which the received signal in FIG. 6C is inverted on the time axis, and the plus-minus sign of the signal level is inverted (a detailed description thereof will be omitted). In other words, as compared with the temporal change of the current at the timing corresponding to the negative coding element of the second driving current, the temporal change of the current at the other timing is smaller. Thus, the temporal change of the second light corresponding to the negative coding element at the reference timing corresponding to the negative coding element is larger than the temporal change of the light intensity at the other timing. The thus-obtained large negative received signal is a received signal corresponding to a negative coding element (for example, a coding element {−1}).

### Rise Time of First Driving Current

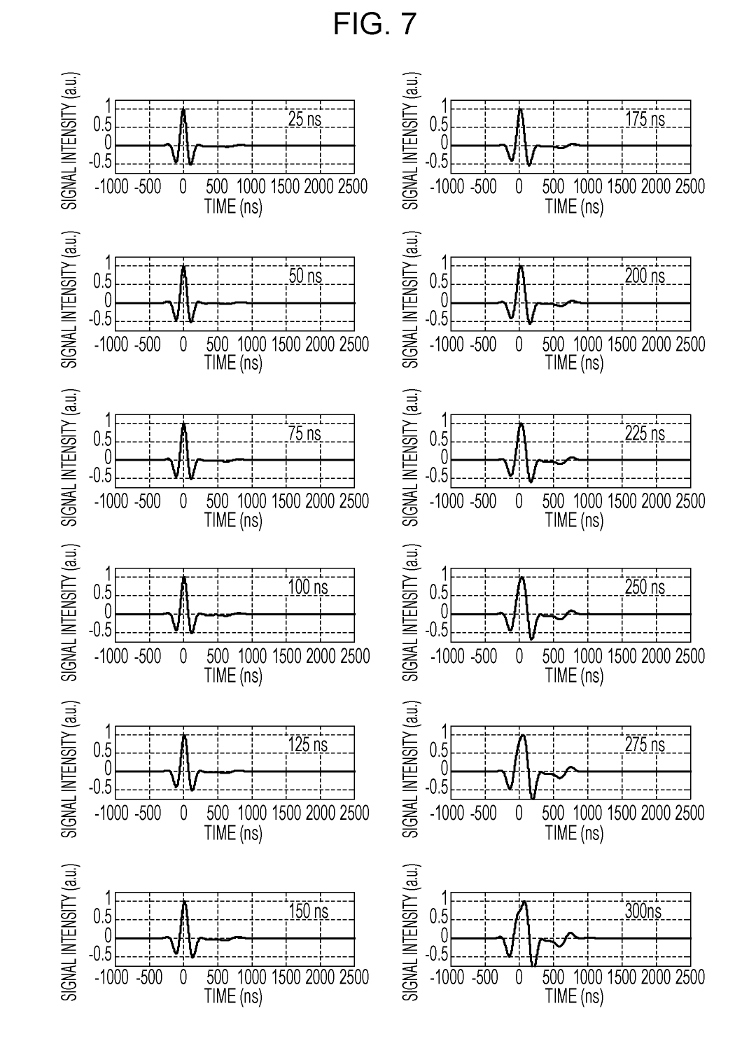

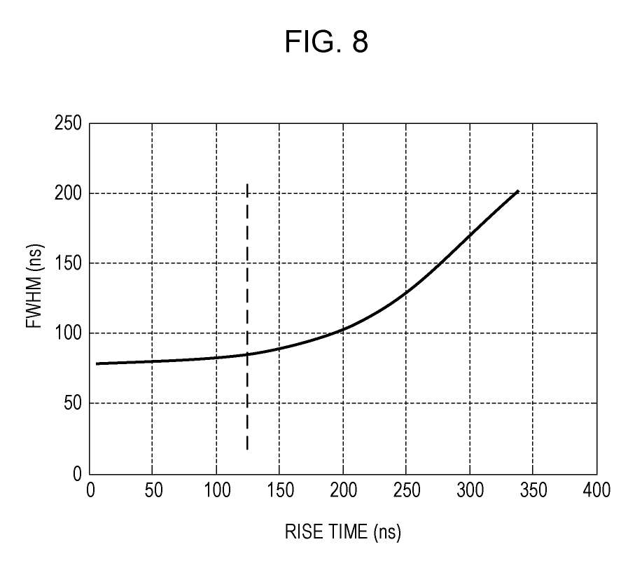

FIG. 5B illustrates an example in which the current value is increased from 0 A to 2 A in the time of 50 ns and is decreased from 2 A to 0 A in the time of 950 ns. The time in which the current value is increased from 0 A to 2 A is referred to as a rise time, and the time in which the current value is decreased from 2 A to 0 A is referred to as a fall time. FIG. 7 illustrates changes of the received signal when the sum of the rise time and the fall time is fixed at 1,000 ns, and the rise time is changed from 25 ns to 300 ns. The transducer has frequency characteristics of a central frequency of 4 MI-Hz and frequencies of 2 to 6 MHz in a 6-dB band, as in FIG. 6B. The values shown at the upper right of the graphs in FIG. 7 indicate the rise time. The relationship between the rise time and the full width at half maximum (FWHM) of the received signal is illustrated in FIG. 8. As illustrated in FIGS. 7 and 8, the full width at half maximum of the received signal increases as the rise time increases. Since the resolution of the image decreases as the full width at half maximum increases, the rise time may be short. In contrast, in this simulation, the rise time may be 125 ns or less because an increase in FWHM can be limited to about 10% when the rise time is 125 ns or less (the dotted line in FIG. 8 indicates 125 ns). The upper limit of the rise time is inversely proportional to the center frequency of the reception band of the transducer. In other words, the rise time is 1/(20 second or less, where f is the center frequency of the reception band of the transducer.

### Fall Time of Second Driving Current

As described above, the received signal of the photoacoustic wave obtained when a semiconductor laser is driven by a driving current corresponding to a negative coding element has a waveform in which the received signal of a photoacoustic wave corresponding to a positive coding element is inversed on the time axis, and the plus-minus sign is inversed. For that reason, the fall time of the second driving current is set to 1/(2f) second or less similarly to the rise time of the first driving current.

The driving unit 113 may apply a driving current in which the rise time of the first driving current or the fall time of the second driving current is 1/(2f) or less to the light source 111. Having described the desirable range of the rise time and fall time of the driving current, this also applies to a temporal change in the intensity of the irradiation light. In other words, to prevent a decrease in resolution, the rise time of light (first light) corresponding to a positive coding element may be set to 1/(2f) or less. To prevent a decrease in resolution, the fall time of light (second light) corresponding to a negative coding element may be set to 1/(2f) second or less.

### Reference Timing of Driving Current

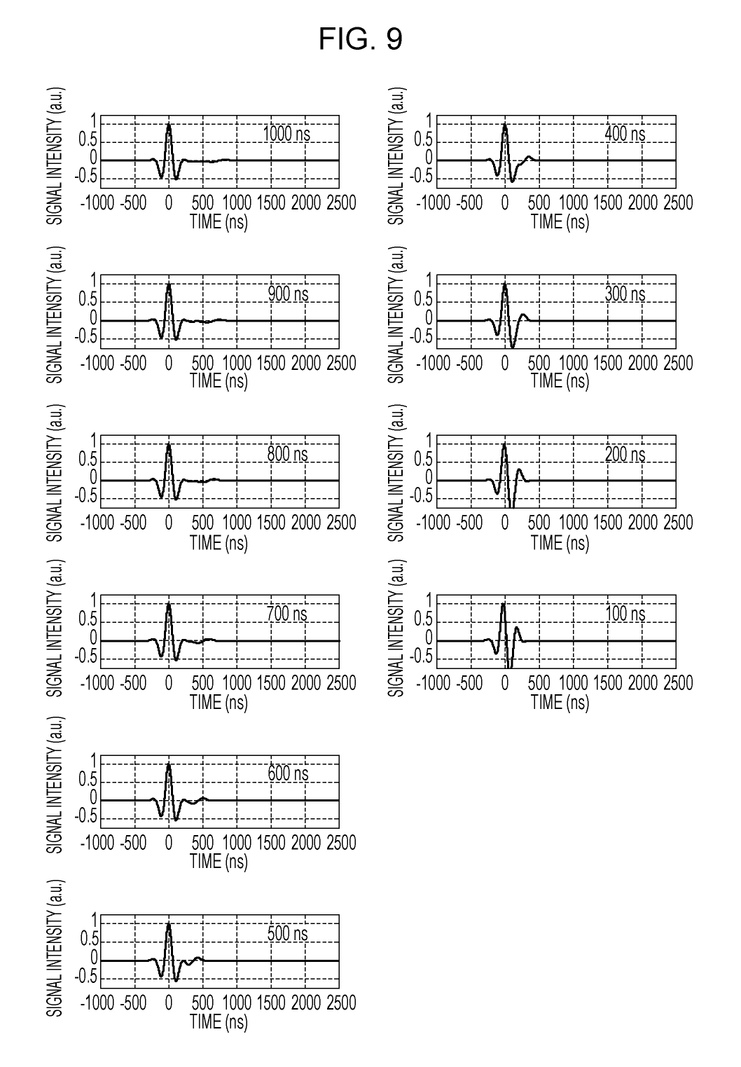

FIG. 9 illustrates changes of the received signal when the rise time is fixed to 50 ns and the sum of the rise time and the fall time is changed in the range from 1,000 ns to 100 ns in the case where the semiconductor laser is driven by the first driving current. The reception characteristic of the transducer is 4 MHz at the center frequency and 2 to 6 MHz at a 6 dB bandwidth, as in FIG. 6B. The values shown at the upper right in the graphs in FIG. 9 each indicate the sum of the rise time and the fall time.

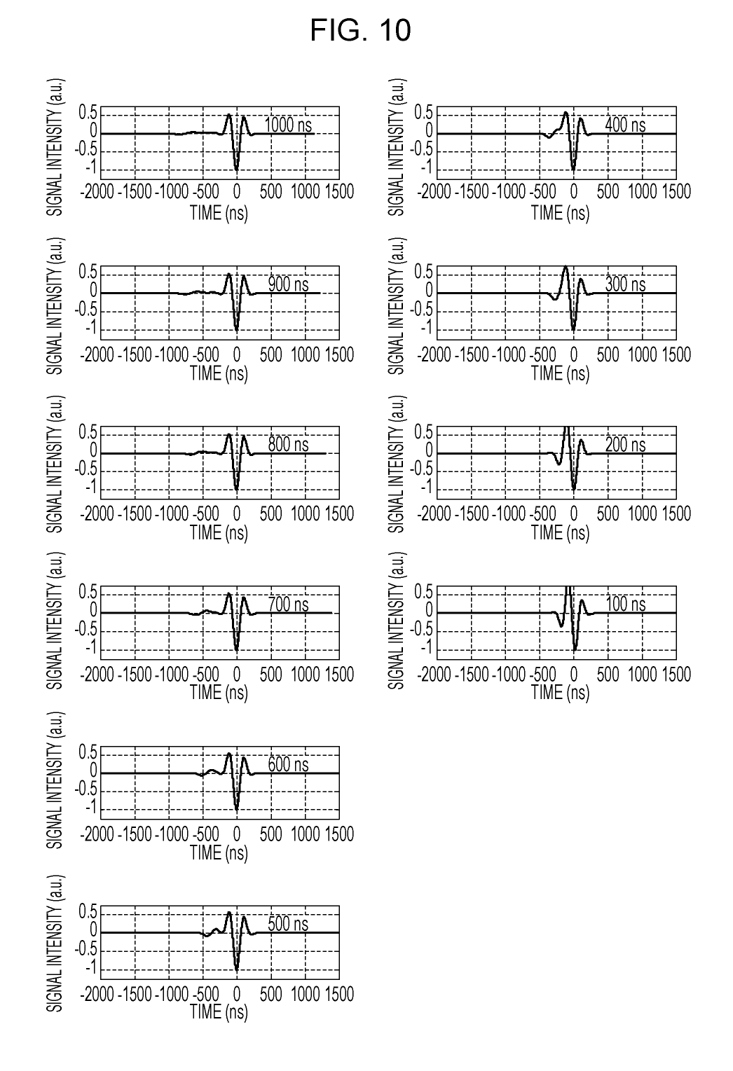

FIG. 10 illustrates changes of the received signal when the fall time is fixed to 50 ns, and the sum of the rise time and the fall time is changed in the range from 1,000 ns to 100 ns in the case where the semiconductor laser is driven by the second driving current. The reception characteristic of the transducer is 4 MHz at the center frequency and 2 to 6 MHz at a 6 dB bandwidth, as in FIG. 6B. The values shown at the upper right in the graphs in FIG. 10 each indicate the sum of the rise time and the fall time.

In FIGS. 9 and 10, the horizontal axis is adjusted so that the time at which the photoacoustic wave generated at the reference timing reaches the transducer is 0.

For a sequence of coding elements, the sum of the rise time and the fall time corresponds to the time interval (period) of the reference timing (the cycle of the coding elements).

Ideally, the received signal corresponding to the coding element {1} and the received signal corresponding to the coding element {−1} are inverted in plus-minus sign. However, the shapes of the received signals come close to each other as the sum of the rise time and the fall time decreases. In other words, the received signal corresponding to the positive coding element and the received signal corresponding to the negative coding element cannot be easily distinguished from each other.

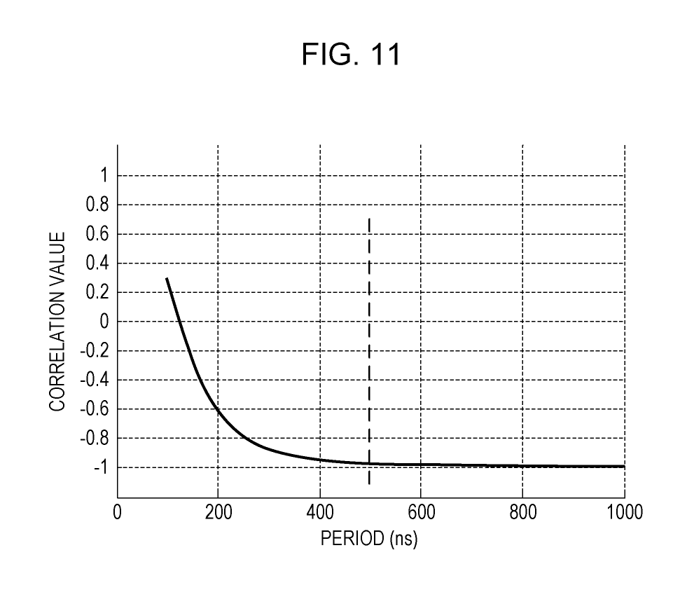

FIG. 11 illustrates the relationship between the sum of the rise time and the fall time (the time interval of the reference timing) and the correlation value of the received signal corresponding to the coding element {1} and the received signal corresponding to the coding element {−1}. In the case where the waveform of the received signal is completely inverted, the correlation value is −1, and the closer to −1, the closer to an ideal state. FIG. 11 shows that the deviation of the correlation value from −1 increases as the sum of the rise time and the fall time (the time interval of the reference timing) decreases. In this example, the sum of the rise time and the fall time (the time interval of the reference timing) may be 500 ns or more (the dotted line in FIG. 11 indicates 500 ns). The lower limit of the time interval of the reference timing is inversely proportional to the center frequency of the reception band of the receiving unit. In other words, the time interval of the reference timing may be 2/f seconds or more, where f is the center frequency of the reception band of the transducer to decrease the correlation value of the received signals corresponding to positive and negative coding elements. The driving unit 113 may apply a driving current that makes the time interval of the reference timing 2/f seconds or more to the light source 111.

### Sequence of Generation of Photographic Image

Next, a method for generating a photographic image by encoding and decoding (a method for acquiring information) using the photoacoustic imaging apparatus according to the present embodiment will be described.

S1. The light irradiation unit 110 applies coded light.

S2. A plurality of transducers in the receiving unit 120 receive photoacoustic waves generated owing to the coded light.

S3. The operating unit 151 decodes individual received signals output from the plurality of transducers to generate a decoded received signal (decoded signal) for each transduce

S4. The operating unit 151 can generate a photographic image using the plurality of decoded signals corresponding to the plurality of transducers.

The coding sequence for use in decoding and encoding may be a known coding sequence, such as a Barker code. A specific method of encoding and decoding will be described later in the embodiments.

### Reconstruction Technique

The operating unit 151 can generate image data by performing back projection (simple back projection) of a plurality of decoded signals on a calculation space. In other words, the operating unit 151 may convert decoded signals, which are time signals, to spatial distribution data. For example, the operating unit 151 may obtain image data (one line of image data) that is linear in a depthwise direction by performing phasing addition on a plurality of decoded signals. The operating unit 151 may generate two-dimensional or three-dimensional image data by executing this process on a plurality of lines. The operating unit 151 may perform envelope processing on the spatial distribution data obtained by the phasing addition to generate image data.

A known PAT image reconstruction method is a universal back projection (UBP) method. This is a method for acquiring a photographic image by back-projecting data obtained by temporally differentiating received signals obtained by the receiving unit and inverting the plus-minus sign. This method can be employed in the case where a photoacoustic wave generated when impulse-like pulsed light is applied has an alphabetical N shape called N-shape.

It can be understood that the photoacoustic wave generated in the present embodiment is a photoacoustic wave in which the former half and the latter half of the N-shape are separated, and the former half is a photoacoustic wave corresponding to the coding element {1}, and the latter half is a photoacoustic wave corresponding to the coding element {−1}. For that reason, the coded and decoded received signals as in the present embodiment cannot yield a correct result even using the UBP method. Therefore, in the present embodiment, back projection may be performed by performing a phasing addition process of phasing and then adding the decoded received signals without performing the preprocessing on the decoded received signals, which is performed in the UBP method. In this specification, the reconstruction method of performing back projection without executing the preprocessing on the decoded received signals in the UBP method is referred to as simple back projection. Other reconstruction algorithms for converting signal data to three-dimensional volume data include back projection in the time domain, back projection in the Fourier domain, a model-based method (a repeated calculation method), and any other methods.

### First Embodiment

### Encoding and Decoding Using Barker Codes

Next, an example of encoding and decoding based on Barker codes will be described. This will be described using the photoacoustic imaging apparatus illustrated in FIG. 3 described above. In the present embodiment, the light source 111 is a semiconductor laser having a wavelength of 780 nm and a maximum light output of 1 W. The receiving unit 120 is a linear array including piezoelectric elements having frequency characteristics of 4 MHz at the center frequency and 2 to 6 MHz at a 6-dB bandwidth. Between the receiving unit 120 and the object 100 is filled with an ultrasonic wave gel for acoustic matching.

In the present embodiment, the control unit 153 transmits information on a Barker code with a code length of 7 to the driving unit 113. It is known that the Barker code is a sequence of codes whose peak value is equal to the code length, and other values are 0 or 1 in the absolute value of its autocorrelation function, and has a code length of 13 or less.

A Barker code with a code length of 7 is {1, 1, 1, −1, −1, 1, −1}.

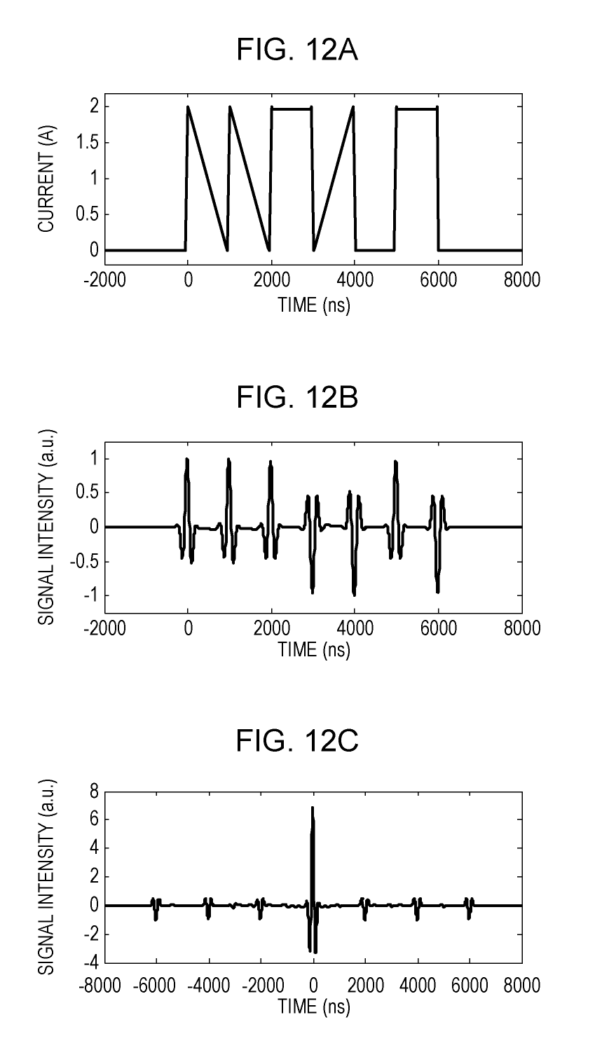

FIG. 12A illustrates the light output waveform of the light source 111 driven by a driving current generated by the driving unit 113 on the basis of information on a Barker code with a code length of 7. In this case, the time interval of the reference timing (corresponding to the cycle of the coding elements) is 1,000 ns. The rise time and the fall time of the first driving current corresponding to the positive coding element are 50 ns and 950 ns, respectively (see FIG. 5B). The rise time and the fall time of the second driving current corresponding to the negative coding element are 950 ns and 50 ns, respectively.

Assuming that a point-like light absorber is present at a depth of 3 mm in the object 100, a received signal obtained by receiving a photoacoustic wave generated when the light illustrated in FIG. 12A is applied to the point sound source with the receiving unit 120 has a waveform as illustrated in FIG. 12B. Actually, a time corresponding to the time the photoacoustic wave is propagated from the point-like light absorber to the receiving unit 120 (about 2 μs) shifts, but the time is ignored in this graph.

A method for decoding a coded received signal with the operating unit 151 in the computer 150 will be described.

The operating unit 151 performs a decoding process according to Expression 1 to obtain a decoded signal DS(t),

$$\begin{matrix}[ Math . 1 ] & \\ DS ( t ) = {\sum }_{i = 1}^{N} S ( t + ( i - 1 ) Δ t ) \cdot {a}_{i} & ( 1 )\end{matrix}$$

where {aᵢ} (i=1 to N, where N is a code length, each coding element is 1 or −1), is a coding sequence, Δt is the time interval of the reference timing, and S(t) is a received signal.

As a result of the process of Expression 1 on the received signal in FIG. 12B, a decoded received signal (a decoded signal) as illustrated in FIG. 12C can be obtained. Thus, it is understood that the peak value of the decoded received signal becomes seven times and the sidelobes are suppressed to 0 or −1.

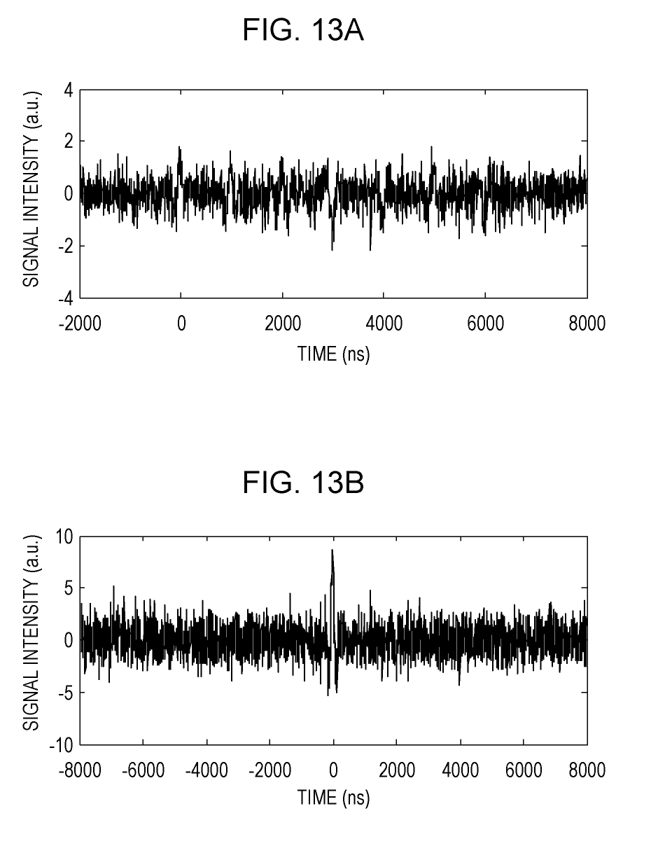

Furthermore, suppose a case where noise with a mean value of 0 and a standard deviation of 0.5 is added to a received signal with a maximum value of 1. FIG. 13A illustrates a received signal in which noise is added to the received signal in FIG. 12A. FIG. 13B illustrates a decoded received signal obtained by performing the decoding process in Expression 1 on the received signal in FIG. 13A. Thus, it is understood that, in the decoded signal in FIG. 13B decoded by using a Barker code, a signal corresponding to the photoacoustic wave is emphasized and noise is suppressed as compared with the received signal in FIG. 13A.

The operating unit 151 can obtain a photographic image in which the signal-to-noise (S/N) ratio is improved by using the thus-obtained decoded signal. If the receiving unit 120 includes a plurality of transducers, a received signal output from each transducer is decoded, so that a decoded signal is generated for each transducer. The operating unit 151 can generate a photographic image using a plurality of decoded signals corresponding to the plurality of transducers with the reconstruction method described above or the like.

In the present embodiment, light irradiation (second light) corresponding to the negative coding element is performed at the time of encoding by light irradiation. This allows high-accuracy encoding on the basis of a coding sequence including a negative coding element (for example, a Barker code). Thus, the present embodiment allows such a coded signal to be decoded with high accuracy by decoding based on a coding sequence containing a negative coding element (for example, the decoding process illustrated in Expression 1). In this manner, the light irradiation corresponding to a negative coding element allows higher-accuracy decoding than that with light irradiation without a negative coding element.

The upper limit of the time interval of the reference timing of coding elements according to the present embodiment will be considered.

The present embodiment uses a code sequence with a code length of 7, so that the signal level of a decoded received signal is seven times, and the noise level is √7 times except sidelobe portions. This increases the S/N ratio by √7 times.

To achieve the same S/N ratio with a generally known method for generating photoacoustic waves by applying impulse light, it is necessary to simply obtain a received signal seven times and average the obtained signals. The time required to obtain one received signal by the general method is equal to the time required for a photoacoustic wave generated a the deepest part of the observation region in the object to reach the receiving unit (the propagation time of light in the object is ignored because it is short). The time required to obtain a received signal seven times is 7T_{tof}, where T_{tof} is the time for one received signal.

In the present embodiment, the time required to obtain a received signal with substantially the same S/N ratio is the sum of the time during which light corresponding to the coding sequence and the time until a photoacoustic wave generated by light corresponding to the last coding element reaches the receiving unit, which is expressed as 6Δt+T_{tof}.

It can be said that the method according to the present embodiment has a higher effect of increasing the S/N ratio than the general method under the condition that the time required to acquire the received signal is short. This condition is expressed as Δt<T_{tof}. In other words, the time interval of the reference timing may be shorter than the time required for the photoacoustic wave generated at the deepest part of the observation region of the object to reach the receiving unit. For example, in the case where the deepest part of the observation region of the object is 5 cm deep, and the sound velocity in the object is 1,500 m/s, the time until the photoacoustic wave generated at the deepest part reaches the receiving unit is 33 μs. In this case, the time interval of the reference timing may be shorter than 33 μs. The control unit 153 may change the time interval of the reference timing according to the region of interest that the user indicates using the input unit 170 so that the time interval is shorter than the time until the photoacoustic wave generated in the deepest part reaches the receiving unit. Alternatively, the control unit 153 may change the time interval of the reference timing according to the instruction of the user or the sound velocity of the object determined by calculation, so that the time interval is shorter than the time until the photoacoustic wave generated in the deepest part reaches the receiving unit. The deepest part is an observation region (the region of interest) farthest from the receiving unit.

### Configuration of Driving Unit

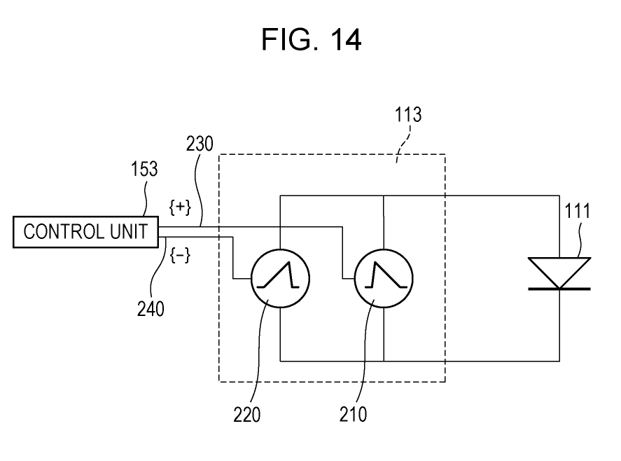

The driving unit 113 may include one power source capable of generating both of the first driving current and the second driving current. Alternatively, the driving unit 113 may include a first power source capable of generating the first driving current and a second power source capable of generating the second driving current. Here, an example in which the driving currents are generated from separate power sources will be described with reference to FIG. 14.

The driving unit 113 illustrated in FIG. 14 includes a first power source 210 capable of generating the first driving current and a second power source 220 capable of generating the second driving current. The control unit 153 has the function of transmitting a first control signal 230 formed of 1 and 0 to the driving unit 113. The control unit 153 also has the function of transmitting a second control signal 240 formed of −1 and 0 to the driving unit 113.

For example, when a Barker code {1, 1, 1, −1, −1, 1, −1} with a code length of 7 is to be generated, the control unit 153 separates the control signal into a first control signal {1, 1, 1, 0, 0, 1, 0} and a second control signal {0, 0, 0, −1, −1, 0, −1} and transmits the control signals to the driving unit 113. In other words, the control unit 153 transmits the first control signal 230 to the first power source 210 and the second control signal 240 to the second power source 220.

The first power source 210 generates the first driving current according to the timing of the coding element {1} and generates no electric current or an electric current in which generation of a photoacoustic wave is suppressed at the timing of coding element {0}. The second power source 220 generates the second driving current according to the timing of the coding element {−1} and generates no electric current or an electric current in which generation of a photoacoustic wave is suppressed at the timing of coding element {0}. As a result, an electric current similar to the driving current (FIG. 12A) corresponding to the Barker code is input to the light source 111.

An apparatus that uses a different power source for each driving current can be simplified in the design of the driving unit 113 as compared with an apparatus that generates different driving currents with a single power source. Using a different power source for each driving current offers high responsivity in high-speed switching between different driving currents. Using a different power source for each driving current when a light source is provided for each driving current allows different driving currents to be input to the separate light sources during the same period. This allows lights with different coding elements to be applied to the object in a temporally overlapping manner. This increases the light irradiation efficiency, allowing decoded signals with high a S/N ratio to be obtained in a short time.

### Second Embodiment

Encoding and Decoding Using Complementary Code

Next, encoding and decoding using complementary codes will be described. The second embodiment will also be described using the same photoacoustic imaging apparatus as that of the first embodiment.

Two coding sequences {aᵢ} and {bᵢ}, (i=1 to N, where N is a code length, each coding element is 1 or −1) with a code length of N in which the sum of their autocorrelation functions are 2N at the peak, and is O at the other all points other than the peak are referred to as complementary codes. For example, a set of {aᵢ}={1, 1} and {bᵢ}={1, −1} are complementary codes. It is known that the complementary codes are present in a code sequence of a code length of n-th power of 2 or n-th power of 10×2 (n is a natural number).

In the present embodiment, complementary codes with a code length of 8 are used. Specifically, {a}={1, −1, 1, −1, −1, −1, 1}) and {bᵢ}={1, −1, −1, −, 1, −1, −1, −1, −1} are used.

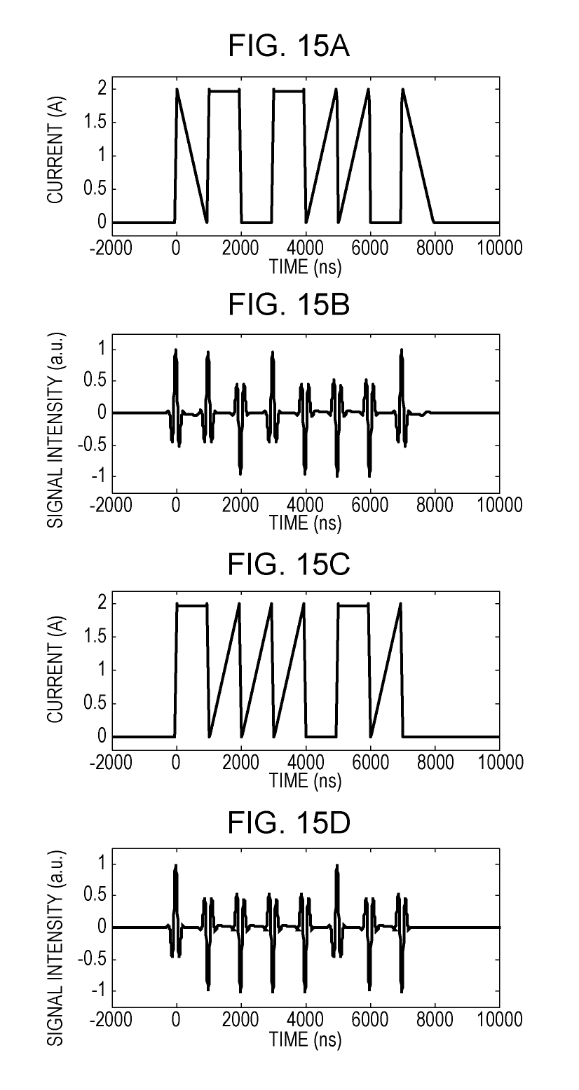

FIG. 15A illustrates the light output waveform of the light source 111 driven by a driving current generated by the driving unit 113 on the basis of information on the coding sequence {aᵢ}. The time interval of the reference timing (corresponding to the cycle of the coding elements) is 1,000 ns. The rise time and the fall time of the first driving current corresponding to the positive coding element are 50 ns and 950 ns, respectively (see FIG. 5B). The rise time and the fall time of the second driving current corresponding to the negative coding element are respectively 950 ns and 50 ns.

Assume that a point-like light absorber is present at a depth of 3 mm in the object 100. A received signal obtained by receiving a photoacoustic wave generated when the light illustrated in FIG. 15A is applied to the point sound source with the receiving unit 120 has a waveform as illustrated in FIG. 15B. Actually, a time corresponding to the time the photoacoustic wave is propagated from the point-like light absorber to the receiving unit 120 (about 2 μs) shifts, but the time is ignored in this graph.

FIG. 15C illustrates the light output waveform of the light source 111 driven by a driving current generated by the driving unit 113 on the basis of information on the coding sequence {bᵢ}. Assume that a point-like light absorber is present at a depth of 3 mm in the object 100. A received signal obtained by receiving a photoacoustic wave generated when the light illustrated in FIG. 15C is applied to the point sound source with the receiving unit 120 has a waveform as illustrated in FIG. 15D. Actually, a time corresponding to the time the photoacoustic wave is propagated from the point-like light absorber to the receiving unit 120 (about 2 μs) shifts, but the time is ignored in this graph.

A method for decoding a coded received signal with the operating unit 151 of in the computer 150 will be described.

The operating unit 151 performs a decoding process according to Expression 2 to obtain a decoded signal DS(t),

$$\begin{matrix}[ Math . 2 ] & \\ DS ( t ) = {\sum }_{i = 1}^{N} Sa ( t + ( i - 1 ) Δ t ) \cdot {a}_{i} + {\sum }_{i = 1}^{N} Sb ( t + ( i - 1 ) Δ t ) \cdot {a}_{i} & ( 2 )\end{matrix}$$

where Δt is the time interval of the reference timing of the code elements, Sa(t) is a receiving signal corresponding to the code sequence {aᵢ}, and Sb(t) is a receiving signal corresponding to the code sequence {bᵢ}.

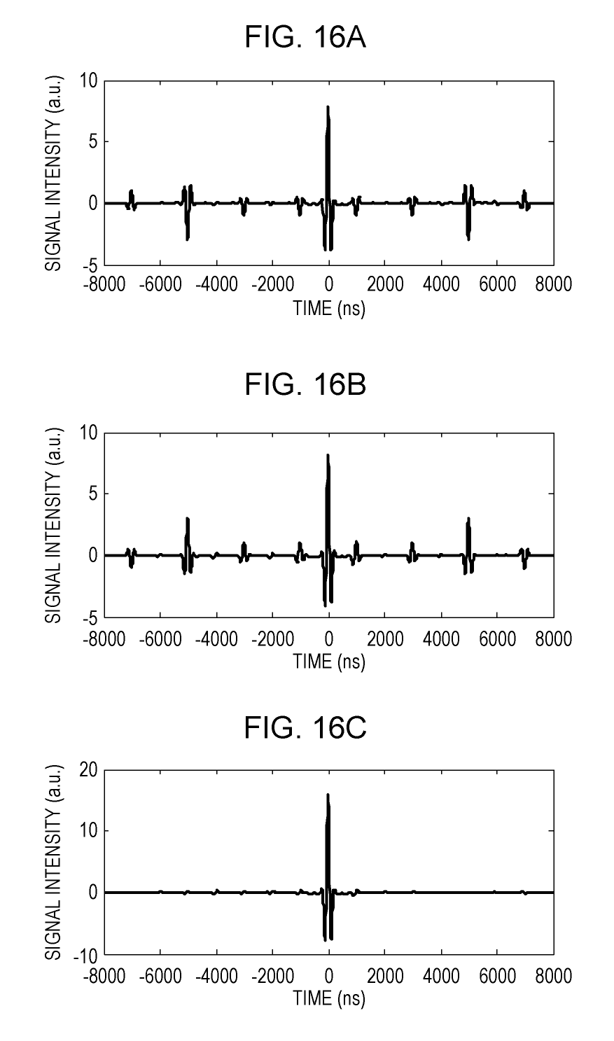

As a result of the decoding process of the first term on the right side of Expression 2 on the reception waveform in FIG. 15B, the decoded received signal as in FIG. 16A can be obtained. As a result of the decoding process of the second term on the right side of Expression 2 on the reception waveform in FIG. 15D, the decoded received signal as in FIG. 16B can be obtained. The sum of the signal in FIG. 16A and the signal in FIG. 16B forms the waveform in FIG. 16C. In other words, the peak value of the received signal decoded according to Expression 2 becomes 16 times, and the sidelobes are suppressed to substantially 0.

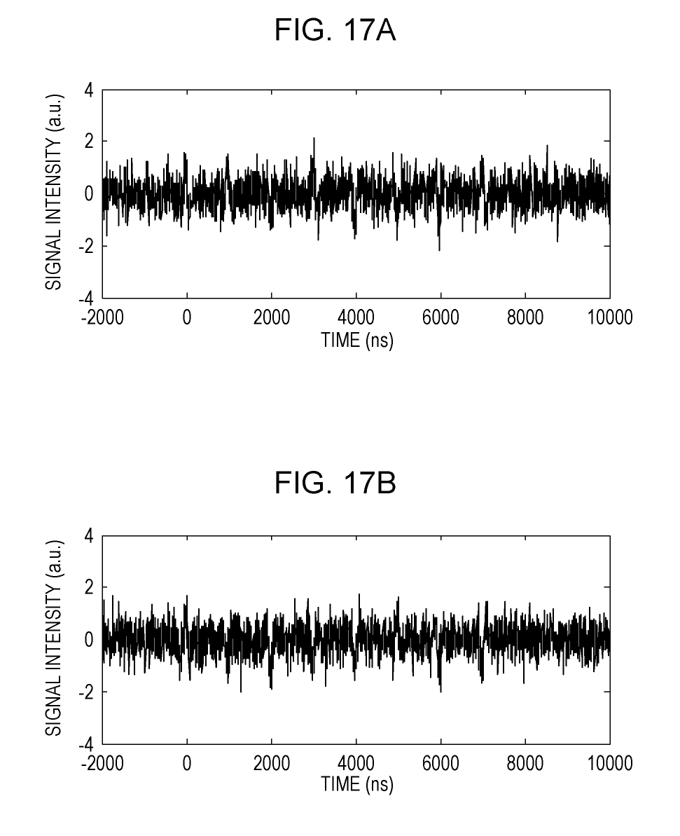

Furthermore, suppose a case where noise with a mean value of 0 and a standard deviation of 0.5 is added to a received signal with a maximum value of 1. FIG. 17A illustrates a received signal corresponding to the code sequence {aᵢ} to which noise is added. FIG. 17B illustrates a received signal corresponding to the code sequence {bᵢ} to which noise is added.

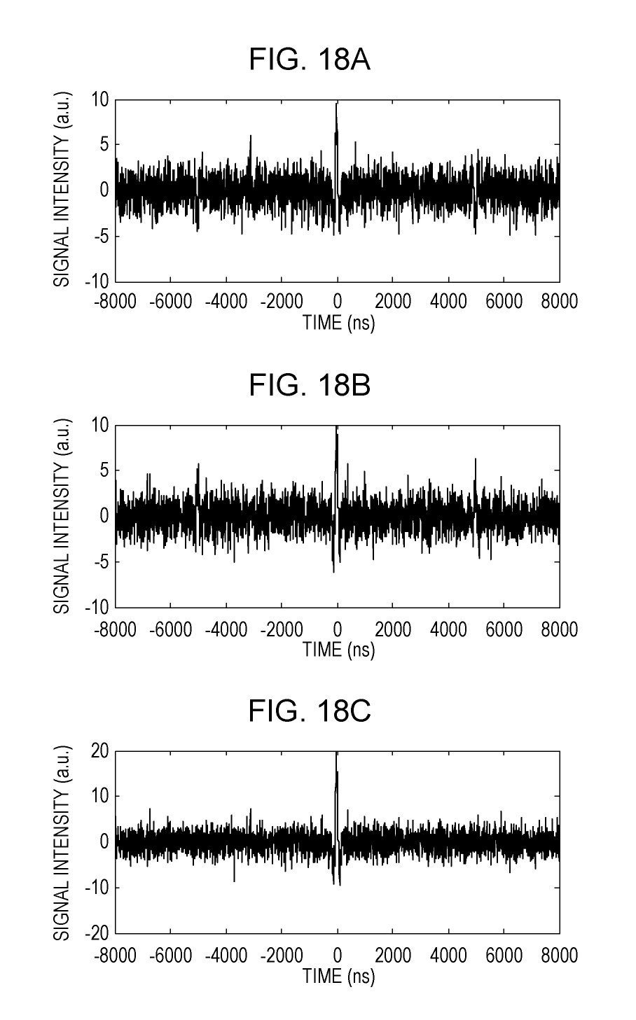

FIG. 18A illustrates a decoded received signal obtained by performing the decoding process of the first term on the right side of Expression 2 on the received signal corresponding to the code sequence {aᵢ} in which noise is added, illustrated in FIG. 17A. FIG. 18B illustrates a decoded received signal obtained by performing the decoding process of the second term on the right side of Expression 2 on the received signal corresponding to the code sequence {bᵢ} in which noise is added, illustrated in FIG. 17B. The sum of the waveform in FIG. 18A and the waveform in FIG. 18B forms the waveform in FIG. 18C. Thus, it is understood that, in the decoded signal in FIG. 18C decoded by using complementary codes, a signal corresponding to the photoacoustic wave is emphasized and noise is suppressed as compared with the received signal in FIG. 17A or 17B.

In the present embodiment, light irradiation (second light) corresponding to the negative coding element is performed at the time of encoding by light irradiation. This allows high-accuracy encoding on the basis of a coding sequence including a negative coding element (for example, a Barker code). Thus, the present embodiment allows such a coded signal to be decoded with high accuracy by decoding based on a coding sequence containing a negative coding element (for example, the decoding process illustrated in Expression 1).

The upper limit of the time interval of the reference timing of coding elements according to the present embodiment will be described.

The present embodiment uses two code sequences with a code length of 8, so that the signal level of a decoded received signal is 16 times, and the noise level is four times. This increases the S/N ratio by four times.

To achieve the same S/N ratio with a generally known method for generating photoacoustic waves by applying impulse light, it is necessary to simply obtain a received signal 16 times and average the obtained signals. The time required to obtain a received signal 16 times by the general method is 16T_{tof} (the propagation time of light in the object is ignored because it is short).

In the present embodiment, the time required to obtain a received signal corresponding to the coding sequence {aᵢ} is the sum of the time during which light corresponding to the coding sequence {aᵢ} and the time until a photoacoustic wave generated by light corresponding to the last coding element reaches the receiving unit. In other words, the time is expressed as 16Δt+T_{tof}. This is the same as the time required to obtain a received signal corresponding to the coding sequence {bᵢ}. Therefore, when a received signal corresponding to the coding sequence {aᵢ} and a received signal corresponding to {bᵢ} are acquired in sequence (in series), the time required to obtain a received signal according to the present embodiment is 14Δt+2T_{tof}. The acquisition of the received signal corresponding to the coding sequence {aᵢ} and the acquisition of the received signal corresponding to {bᵢ} may be executed such that at least part is temporally overlapped. Also in this case, the received signal corresponding to the coding sequence {aᵢ} and the received signal corresponding to {bᵢ} can be separately decoded, allowing a decoded signal in which a signal corresponding to a photoacoustic wave is emphasized to be obtained.

As in the first embodiment, the control unit 153 may set the time interval of the reference timing according to the observation region (the region of interest) or the sound velocity of the object so that a signal with a high S/N ratio can be obtained in a short time. A method of reconstruction may be the same method as that of the first embodiment. The present embodiment may also employ the configuration of the driving unit 113 described in the first embodiment.

In another aspect, the present disclosure is the method of light irradiation corresponding to a coding sequence including a negative coding element, described in the above embodiments.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-209922, filed Oct. 26, 2016, which is whereby incorporated by reference herein in its entirety.

1. A photoacoustic imaging apparatus comprising:
a light irradiation unit;
a receiving unit; and
a processing unit,
wherein the light irradiation unit irradiates an object with first light for generating a photoacoustic wave corresponding to a positive coding element constituting a coding sequence and irradiates the object with second light for generating a photoacoustic wave corresponding to a negative coding element constituting the coding sequence,
wherein the receiving unit outputs a first signal by receiving the photoacoustic wave corresponding to the positive coding element, the photoacoustic wave being generated by irradiating the object with the first light, and outputs a second signal by receiving the photoacoustic wave corresponding to the negative coding element, the photoacoustic wave being generated by irradiating the object with the second light, and
wherein the processing unit obtains a decoded signal by performing a decoding process on the first signal and the second signal based on information on the coding sequence.

2. The photoacoustic imaging apparatus according to claim 1,
wherein a temporal change in light intensity of the first light at a reference timing corresponding to the positive coding element is larger than a temporal change in light intensity at another timing, and
wherein a temporal change in light intensity of the second light at a reference timing corresponding to the negative coding element is larger than a temporal change in light intensity at another timing.

3. The photoacoustic imaging apparatus according to claim 1, wherein rise time of the reference timing corresponding to the positive coding element of the first light is 1/(2f) seconds or less, where f is a center frequency of a reception band of the receiving unit.

4. The photoacoustic imaging apparatus according to claim 1, wherein fall time of the reference timing of corresponding to the negative coding element of the first light is 1/(2f) seconds or less, where f is a center frequency of a reception band of the receiving unit.

5. The photoacoustic imaging apparatus according to claim 1, wherein a time interval between irradiation timing of the first light and irradiation timing of the second light is equal to or longer than 2/f seconds, where f is the center frequency of the reception band of the receiving unit.

6. The photoacoustic imaging apparatus according to claim 1, wherein, when the positive coding element and the negative coding element of the coding sequence are next to each other, the light irradiation unit sets light intensity between the reference timing corresponding to the positive coding element and the reference timing corresponding to the negative coding element to fall within a predetermined range.

7. The photoacoustic imaging apparatus according to claim 6, wherein, when the positive coding element and the negative coding element of the coding sequence are next to each other, the light irradiation unit sets a waveform of the temporal change in the light intensity to a rectangular wave.

8. The photoacoustic imaging apparatus according to claim 1,
wherein the coding sequence is a coding sequence according to a Barker code, and
wherein the processing unit acquires the decoded signal DS(t) by performing the decoding process according to a following equation,
$$DS ( t ) = {\sum }_{i = 1}^{N} S ( t + ( i - 1 ) Δ t ) \cdot {a}_{i} ,$$
where {aᵢ} (i=1 to N, where N is a code length, and each coding element is 1 or −1) is the coding sequence, Δt is a time interval between the irradiation timing of the first light and the irradiation timing of the second light, and S(t) is a signal including the first signal and the second signal.

9. The photoacoustic imaging apparatus according to claim 1,
wherein the coding sequence includes a first coding sequence and a second coding sequence that differ from each other and have a complementary relationship, and
wherein the processing unit acquires the decoded signal DS(t) by performing the decoding process according to a following equation,
$$DS ( t ) = {\sum }_{i = 1}^{N} Sa ( t + ( i - 1 ) Δ t ) \cdot {a}_{i} + {\sum }_{i = 1}^{N} Sb ( t + ( i - 1 ) Δ t ) \cdot {b}_{i} ,$$
where {aᵢ} is the first coding sequence, {bᵢ} is the second coding sequence (i=1 to N, where N is a code length, each coding element is 1 or −1), Δt is a time interval between the irradiation timing of the first light and the irradiation timing of the second light, Sa(t) is a signal including the first signal and the second signal corresponding to the first coding sequence, and Sb(t) is a signal including the first signal and the second signal corresponding to the second coding sequence.

10. The photoacoustic imaging apparatus according to claim 1,
wherein the light irradiation unit comprises:
a light source comprising a semiconductor laser or a light-emitting diode; and
a driving unit configured to input a first driving current or a second driving current to the light source,
wherein the driving unit applies the first light to the object by inputting the first driving current to the light source at the reference timing corresponding to the positive coding element, and
wherein the driving unit applies the second light to the object by inputting the second driving current to the light source at the reference timing corresponding to the negative coding element.

11. The photoacoustic imaging apparatus according to claim 10,
wherein the light source comprises:
a first light source comprising a semiconductor laser or a light-emitting diode that generates the first light; and
a second light source comprising a semiconductor laser or a light-emitting diode that generates the second light, and
wherein the driving unit comprises:
a first driving unit configured to input the first driving current for causing the first light source to generate the first light; and
a second driving unit configured to input the second driving current for causing the second light source to generate the second light.

12. The photoacoustic imaging apparatus according to claim 1,
wherein the receiving unit comprises a plurality of transducers,
wherein each of the plurality of transducers outputs the first signal and the second signal, and
wherein the processing unit obtains a plurality of the decoded signals corresponding to the plurality of transducers by performing the decoding process on each of the plurality of the first signals and the second signals corresponding to the plurality of transducers, and acquires image data based on the plurality of decoded signals corresponding to the plurality of transducers.

13. The photoacoustic imaging apparatus according to claim 12, wherein the processing unit acquires the image data by simply back-projecting the plurality of decoded signals corresponding to the plurality of transducers.

14. A photoacoustic imaging apparatus comprising:
a light irradiation unit configured to irradiate an object with intensity-modulated light including light corresponding to a positive coding element and light corresponding to a negative coding element;
a receiving unit configured to output a signal by receiving a photoacoustic wave generated by irradiating the object with the intensity-modulated light; and
a processing unit configured to obtain a decoded signal by performing a decoding process on the signal.

15. A method for acquiring information, the method comprising:
irradiating an object with first light for generating a photoacoustic wave corresponding to a positive coding element constituting a coding sequence;
irradiating the object with second light for generating a photoacoustic wave corresponding to a negative coding element constituting the coding sequence;
obtaining a first signal by receiving the photoacoustic wave corresponding to the positive coding element, the photoacoustic wave being generated by irradiating the object with the first light;
obtaining a second signal by receiving the photoacoustic wave corresponding to the negative coding element, the photoacoustic wave being generated by irradiating the object with the second light; and
obtaining a decoded signal by performing a decoding process on the first signal and the second signal based on information on the coding sequence.

16. A method for acquiring information, the method comprising:
irradiating an object with intensity-modulated light including light corresponding to a positive coding element and light corresponding to a negative coding element;
obtaining a signal by receiving a photoacoustic wave generated by irradiating the object with the intensity-modulated light; and
obtaining a decoded signal by performing a decoding process on the signal.

17. A non-transitory computer-readable medium storing a program for causing a computer to execute the method for acquiring information according to claim 15.

18. A non-transitory computer-readable medium storing a program for causing a computer to execute the method for acquiring information according to claim 16.
